SEQUENCE LISTING (4 sequences)

SEQ ID NO: 1 - protein, 30 aa, Artificial Sequence
  chemically synthesized
NGMIGEVVYQRAVMAVGSLTIKRIVTEKTD

SEQ ID NO: 2 - protein, 19 aa, Artificial Sequence
  chemically synthesized
DLLEKIAEDRVEFNEDGDR

SEQ ID NO: 3 - protein, 23 aa, Artificial Sequence
  chemically synthesized
WSLRRDPRGAPAWARGSRPRHAS

SEQ ID NO: 4 - protein, 22 aa, Artificial Sequence
  chemically synthesized
TINEERSEIVWSLRRDPRGAPA

(19) United States
(12) Patent Application Publication
(10) Pub. No.: US 2012/0083586 A1
(43) Pub. Date: Apr. 5, 2012

(54) NMDAR BIOMARKERS FOR DIAGNOSING AND TREATING CEREBRAL ISCHEMIA

(75) Inventor: Svetlana Dambinova, Atlanta, GA (US)

(73) Assignee: CIS BIOTECH, INC., Atlanta, GA (US)

(21) Appl. No.: 13/293,935

(22) Filed: Nov. 10, 2011

Related U.S. Application Data
(63) Continuation of application No. 12/518,629, filed on Jun. 10, 2009, now abandoned.
(60) Provisional application No. 60/874,356, filed on Dec. 12, 2006.

(51) Int. Cl. C07K 14/705; C07H 21/00; C07K 7/08

(52) U.S. Cl. 530/324; 530/326; 536/23.5

(57) ABSTRACT

The present invention relates to methods for detecting various subunits and isoforms of NMDA receptors to help diagnose and differentiate (1) the anatomical location of NMDA receptor over-expression, and (2) ischemic conditions in the central and peripheral nervous systems.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds of the invention may contain charged side chains or termini, they may be included in any of the above-described formulations as the free bases or as pharmaceutically acceptable salts. Pharmaceutical acceptable salts are those salts which substantially retain the biologic activity of the free bases and which are prepared by reaction with inorganic acids. Pharmaceutical salts tend to be more soluble in aqueous and other protic solvents than are the corresponding free base forms.

The compounds of the invention will generally be used in an amount effective to achieve the intended purpose (e.g., treatment of central or peripheral neuronal injury). The therapies of the invention are performed by administering the subject drug in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein. An “therapeutic amount” or “therapeutic concentration” of a NMDAR isoforms or antibodies is an amount that reduces binding activity to receptor by at least about 40%, preferably at least about 50%, often at least about 70%, and even as much as at least about 90%. Binding can be measured in vitro (e.g., in an assay ) or in situ.

For systemic administration, a therapeutically effective dose can be estimated initially from in vitro assays. For example, a dose can be formulated in animal models to achieve a circulating concentration range that includes the IC50. Such information can be used to more accurately determine useful doses in humans.

Initial dosages can also be estimated from in vivo data, e.g., animal models, using techniques that are well known in the art. One having ordinary skill in the art could readily optimize administration to humans based on animal data. Dosage amount and interval may be adjusted individually to provide plasma levels of the compounds that are sufficient to maintain therapeutic effect. Usual patient dosages for administration by injection range from about 0.1 to 10 mg/day, preferably from about 0.5 to 1 mg/day. Therapeutically effective serum levels may be achieved by administering multiple doses each day. For usual peptide/antibodies therapeutic treatment of cerebral ischemic events within 6 hours of event is typical.

In cases of local administration or selective uptake, the effective local concentration of the compounds may not be related to plasma concentration and should be optimized therapeutically effective local dosages without undue experimentation. The amount of compound administered will, of course, be dependent on the subject being treated, on the subject's weight, the severity of the affliction, the manner of administration and the judgment of the prescribing physician. The therapy may be repeated intermittently while symptoms detectable or even when they are not detectable. The therapy may be provided alone or in combination with other drugs.

Preferably, a therapeutically effective dose of the compounds described herein will provide therapeutic benefit without causing substantial toxicity. Toxicity of the compounds described herein can be determined by standard pharmaceutical procedures in experimental animals, e.g., by determining the LD50 (the dose lethal to 50% of the population) or the LD100 (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics. Ch. 1, p. 11.

### Diagnostic Platforms

The diagnostic methods of the present invention can be performed using any number of known diagnostic techniques, including direct or indirect ELISA, RIA, immunodot, immunoblot, latex aggutination, lateral flow, fluorescence polarization, and microarray. In one particular embodiment, the invention is practiced using an immobilized solid phase for capturing and measuring the NMDAR peptide marker. Therefore, in one embodiment the methods of the invention comprise: (a) contacting a biological sample from the patient with an immobilized solid phase comprising a NMDAR peptide or antibody, for a time sufficient to form a complex between said NMDAR peptide or antibody and NMDAR antibody or peptide in said biological sample; (c) contacting said complex with an indicator reagent attached to a signal-generating compound to generate a signal; and (d) measuring the signal generated. In a preferred embodiment, the indicator reagent comprises chicken anti-human or anti-human IgC attached to horseradish peroxidase.

In a preferred embodiment, the solid phase is a polymer matrix. More preferably, the polymer matrix is polyacrylate, polystyrene, or polypropylene. In one preferred embodiment the solid phase is a microplate. In another preferred embodiment, the solid phase is a nitrocellulose membrane or a charged nylon membrane.

In another embodiment, the method is performed using agglutination. Therefore, in still another embodiment the invention comprises: (a) contacting a biological sample from the patient with an agglutinating carrier comprising a NMDAR peptide or antibody, for a time sufficient to form an agglutination complex between said NMDAR peptide or antibody and NMDAR antibody or peptide in said biological sample; (c) generating a signal from the agglutination: (d) correlating said signal to said levels of one or more markers of NMDAR peptide or antibody. In a preferred embodiment, the “sufficient lime” is less than 30, 20, 15 or even 10 minutes.

Latex agglutination assays have been described in Beltz, G. A. et al., in Molecular Probes: Techniques and Medical Applications, A. Albertini et al., eds., Raven Press, New York, 1989, incorporated herein by reference. In the latex agglutination assay, antibody raised against a particular biomarker is immobilized on latex particles. A drop of the latex particles is added to an appropriate dilution of the serum to be tested and mixed by gentle rocking of the card. With samples lacking sufficient levels of the biomarkers, the latex particles remain in suspension and retain a smooth, milky appearance. However, if biomarkers reactive with the antibody are present, the latex particles clump into visibly detectable aggregates.

An agglutination assay can also be used to detect biomarkers wherein the corresponding antibody is immobilized on a suitable particle other than latex beads, for example, on gelatin, red blood cells, nylon, liposomes, gold particles, etc. The presence of antibodies in the assay causes agglutination, similar to that of a precipitation reaction, which can then be detected by such techniques as nephelometry, turbidity, infrared spectrometry, visual inspection, colorimetry, and the like.

The term latex agglutination is employed generically herein to refer to any method based upon the formation of detectable agglutination, and is not limited to the use of latex as the immunosorbent substrate. While preferred substrates for the agglutination are latex based, such as polystyrene and polypropylene, particularly polystyrene, other well-known substrates include beads formed from glass, paper, dextran, and nylon. The immobilized antibodies may be covalently, ionically, or physically bound to the solid-phase immunoadsorbent, by techniques such as covalent bonding via an amide or ester linkage, ionic attraction, or by adsorption. Those skilled in the art will know many other suitable carriers for binding antibodies, or will be able to ascertain such, using routine experimentation.

Conventional methods can be used to prepare antibodies for use in the present invention. For example, by using a peptide of a NMDA protein, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be administered and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor el al., Immunol. Today 4, 72 (1983)), the EBV-hybridoma technique to produce human monoclonal antibodies (Cole et al. Monoclonal Antibodies in Cancer Therapy (1985) Allen R. Bliss, Inc. pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for NMDAR proteins or fragments as described herein.

In one embodiment the method is practiced using a kit that has been calibrated at the factory based upon antibodies purified from human blood. Therefore, in another embodiment the invention is practiced under the following conditions: (a) NMDAR antibody levels in said biological fluid are measured using a diagnostic kit; (b) said diagnostic kit comprises bound NMDAR peptides; and (c) said kit is manufactured against an antibody standard comprising a fraction of immunoglobulins G purified from human blood.

In addition, the method can be practiced using commercially available chemiluminescence techniques. For example, the method could employ a two-site sandwich immunoassay using direct chemiluminescent technology, using constant amounts of two monoclonal antibodies. The first antibody, in a fluid reagent, could be an acridinium ester labeled monoclonal mouse anti-human NMDA receptor peptide BNP (F(ab′)₂ fragment specific to a first portion of the peptide. The second antibody, in the solid phase, could be a biotinylated monoclonal mouse anti-human antibody specific to another portion of the peptide, which could be coupled to streptavidin magnetic particles. An immuno-complex would be formed by mixing a patient sample and the two antibodies. After any unbound antibody conjugates are washed away, the chemiluminescence of the immuno-complex signal could then be measured using a luminometer.

When the NMDA receptors are detected indirectly, by measuring the cDNA expression of the NMDA receptors, the measuring step in the present invention may be carried out by traditional PCR assays such as cDNA hybridization, Northern blots, or Southern blots. These methods can be carried out using oligonucleotides encoding the polypeptide antigens of the invention. Thus, in one embodiment the methods of this invention include measuring an increase of NMDAR cDNA expression by contacting the total DNA isolated from a biological sample with oligonucleotide primers attached to a solid phase, for a sufficient time period. In another preferred embodiment, NMDAR cDNA expression is measured by contacting an array of total DNA bound to a solid matrix with a ready-to-use reagent mixture containing oligonucleotide primers for a sufficient time period. Expressed NMDAR cDNA is revealed by the complexation of the cDNA with an indicator reagent that comprises a counterpart oligonucleotide to the cDNA attached to a signal-generating compound. The signal-generating compound is preferably selected from the group consisting of horseradish peroxidase, alkaline phosphatase, urinase and non-enzyme reagents. The signal-generating compound is most preferably a non-enzyme reagent.

The immunosorbent of the present invention for measuring levels of autoantibody can be produced as follows. A fragment of the receptor protein is fixed, preferably by covalent bond or an ionic bond, on a suitable carrier such as polystyrene or nitrocellulose. If the standard polystyrene plate for immunological examinations is employed, it is first subjected to the nitration procedure, whereby free nitrogroups are formed on the plate surface, which are reduced to amino groups and activated with glutaric dialdehyde serving as a linker. Next the thus-activated plate is incubated with about 2 to 50 nM of the target peptide for the purpose of chemically fixing the respective immunogenic fragment of the receptor protein for a time and at a temperature sufficient to assure fixation (i.e. for about 16 hours at 4° C).

It is also practicable to produce the immunosorbent by fixing the respective fragment of the receptor protein on nitrocellulose strips by virtue of ionic interaction. The respective fragment of the receptor protein isolated from the mammals' brain is applied to nitrocellulose and incubated for 15 min at 37° C. Then nitrocellulose is washed with a 0.5 % solution of Tween-20, and the resultant immunosobent is dried at room temperature and stored in dry place for one year period.

### EXAMPLES

The following examples are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, pans are parts by weight, temperature is in ° C. or is at room temperature, and pressure is at or near atmospheric.

### Example 1

### Combined Use of NMDAR Test and Antiplatelet Therapy

The FDA recently approved the use of aspirin to prevent stroke in men and women who have already had a stroke or mini-stroke to decrease the likelihood of a second stroke. A large meta-analysis showed that aspirin reduces the incidence of stroke, myocardial infarction, or vascular death by 25%. (Collaborative overview of randomized trials of antiplatelet therapy 1: Prevention of death, myocardial infarction and stroke by prolonged antiplatelet therapy in various categories of patients. Antiplatelet Trialists Collaboration. BMJ 1994; 308:81-106.)

Aspirin also is an established treatment for the prevention of cerebrovascular accidents (CVA) in patients with transitory ischemic attacks (TIA) or minor cerebral vascular accident (CVA). It reduces the risk of TIA by 20%. (Kase. C S. Antiaggregant treatment: present and future Rev Neurol. 1999; 28:1013-6 Article in Spanish). However some patients still have a vascular event even if they are taking aspirin. Researchers have found that some patients are predisposed to biochemical aspirin resistance. (Gum P A, Marchant K K. Welsh P A. et al. A prospective, blinded determination of the natural history of aspirin resistance among stable patients with cardiovascular disease. J Am Coll Cardiol 2003; 41:961-965.)

In this study, serial measurements of blood levels of NR2 antibodies in an out-patient setting in individuals with diabetes mellitus (DM) and atherosclerosis (AS) who are at high risk for subsequent CVA were evaluated to establish whether this biomarker can be used to monitor the efficacy of aspirin treatment. Every person who participated in the study underwent neurological examination and computed tomography (CT) of the brain. The age of the 33 persons (17 male/16 female) ranged from 47-64 yrs (mean 55.7 yrs). Patients with documented 5-10 year history of DM/AS and normal neurological examination without a previous history of neurological event such as transient schemic attack (TIA), or prolonged reversible ischemic neurological deficit or stroke were observed. Based on laboratory and clinical testing, all participants were at borderline glucose intolerance, with blood glucose levels of 119 to 132 mg/dL, and had complaints on fatigue, low concentration of attention, and numbness in digits of left or right hand.

Out of 22 individuals, 10 (6 male/4 female) were randomly assigned to a placebo group and the rest used daily over-counter aspirin in a dose of 325 mg which formed group 1 (Table 3). None of people of group 1 developed the resistance to aspirin. Group 2 individuals with DM/AS (n=9) who were resistant to aspirin (325 mg/day) were revealed from a retrospective study of 186 persons at high risk tor subsequent CVA performed at Clinic of Pavlov' Medical University (St. Petersburg, Russia) in 2001-2002.

TABLE 3
Cumulative detection of NR2 antibodies over 24 months of aspirin treatment follow up

| | | NR2 antibodies (ng/ml) over 24 months of follow up | | | | | TIA |
|---|---|---|---|---|---|---|---|
| Group | N | 0 mo. | 6 mo. | 12 mo. | 18 mo. | 24 mo. | rate |
| Placebo | 10 | 2.91 ± 0.35 | 2.84 ± 0.12 | 2.77 ± 0.19 | 2.95 ± 0.19 | 3.21 ± 0.45 | 1/10 |
| Group 1 | 12 | 3.05 ± 0.21 | 2.51 ± 0.28 | 2.33* ± 0.32 | 1.92* ± 0.25 | 1.60** ± 0.25 | 0 |
| Group 2 | 9 | 2.87 ± 0.11 | 3.22 ± 0.30 | 3.51* ± 2.2 | 4.05* ± 0.21 | 5.14** ± 0.33 | 2/9 |

*P < 0.01 and **P < 0.001 compared with basic level of placebo and corresponding group.

NR2 antibodies were detected at a base level for each group before treatment was initiated (0 mo.) and then each following 6 months for two years. All three groups of individuals demonstrated close initial levels of NR2 antibodies that exceeded the cut off of 2 ng/ml by about 43.5-52.5%%. Follow up after treatment of the placebo group showed insignificant alterations in antibodies contents during the 24 months of treatment. A TIA event was documented in 1 patient from the placebo group. The levels of NR2 antibodies were significantly reduced (below cut off of 2 ng/ml) in patients in group 1 with no CVA consequences in 1 year of treatment follow up. Patients from group 2 resistant to aspirin had no improvement in NR2 antibodies levels at any time during the study. By the end of the study at 24 mo., two out of 9 persons suffered a TIA which developed into an acute ischemic stroke in one patient.

### Example 2

### Combined Use of NMDAR Test and Pentoxifylline Therapy

Trental (pentoxifylline) is an antiplatelet drug that improves oxygen supplies to tissues by stimulating vasodilation and inhibiting platelet aggregation. It reduces blood viscosity and the risk of a new blood clot forming in the brain.

There is some evidence for the effectiveness of the Trental in the routine management of acute ischemic stroke (non-significant reduction of odds of early death—odds ratio 0.64; 95% C1: 0.41, 1.02). No data on outcomes such as quality of life or stroke recurrence are available. (Bath P M W. Bath S J. Asplund K. Pentoxifylline, propentofylline and pentifylline in acute ischaemic stroke. Cochrane Review [Updated 11 Jun. 1996). In: The Cochrane Library, Issue 4. Oxford: Update Software, 1998).

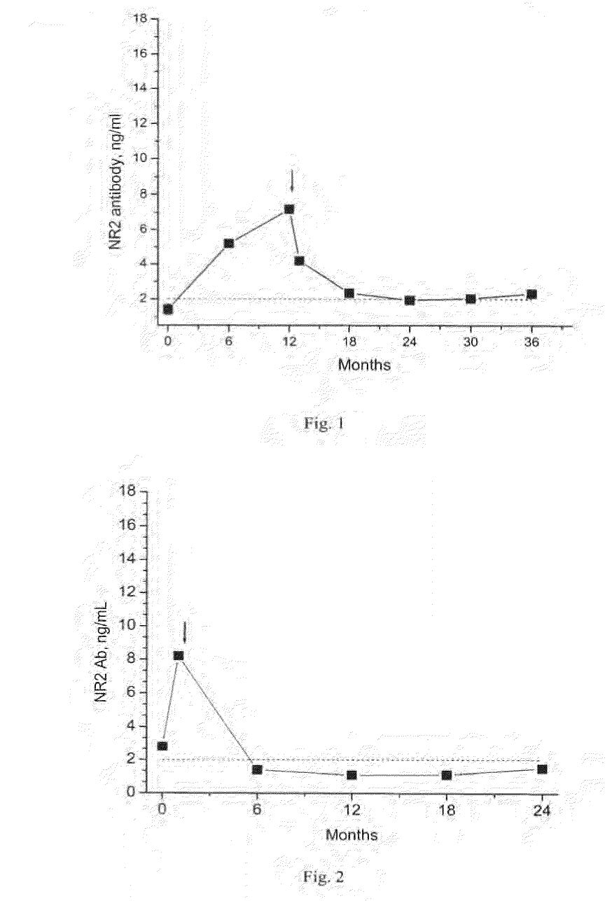

We observed three patients with minor CVA, TIA and ischemic stroke to demonstrate the efficacy of trental as a preventive agent for TIA/stroke. The first case was a 63 -year-old male non-smoker who had previously undergone valve replacement surgery in 1987 and had received a coronary artery stent in 2002. The patient had been treated with coumudin since the first surgery. NR2 antibodies measured at base line were below the 2 ng/ml cutoff. (FIG. 1). Repeated blood assessments revealed a sudden increase of NR2 antibodies that showed a tendency toward further increases. At the same time, the patient had complaints on chronic fatigue and numbness in the 4^{th} and 5^{th} digits of his left arm lasting quite a long period of time. Oral trental taken in 200 mg twice a day was initiated for 30 days, beginning at the point designated by an arrow in FIG. 1, and reduced the level of NR2 antibodies by the end of treatment. Antibodies continued to decrease up to 6^{th} mo. after treatment was initiated and remained below the cut off level more than 1 year after therapy.

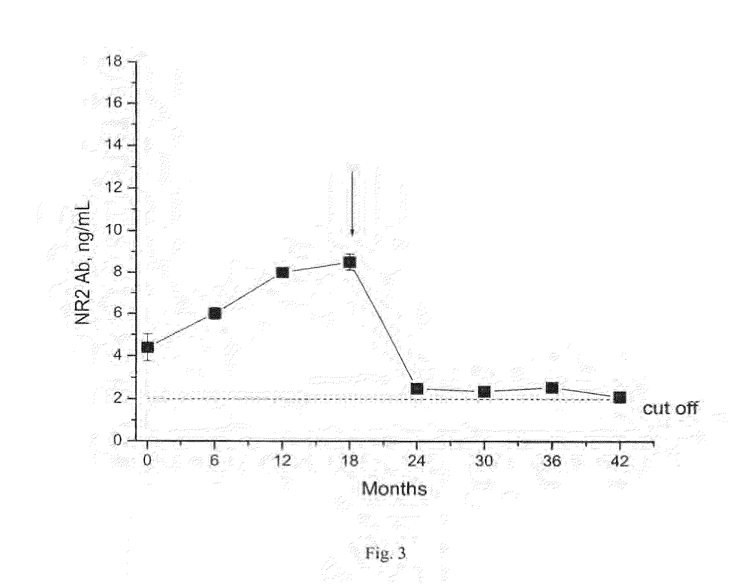

The second patient was a 66-year-old apparently healthy woman. Her blood assay for NR2 antibodies was performed initially on a voluntary basis as a healthy control. Slightly increased antibody levels above the 2.0 ng/ml cutoff were detected 1 mo before TIA onset (March 2002) that resulted in disorientation, and loss of consciousness for about 1 hour. DWI performed a week after TIA onset noted an asymmetry of the internal artery, neurological observation; EEG, EKG, and carotid ultrasound showed no abnormalities. However, NR2 antibodies detected within a week of onset were drastically increased (FIG. 2). Oral trental taken in 200 mg twice a day for 30 days reduced the level of NR2antibodies to the control level of healthy individuals. Antibody monitoring for 1 year showed steady normal amounts that remains the same up to 2005. The patient used Plavix and Lipitor on daily basis as well.

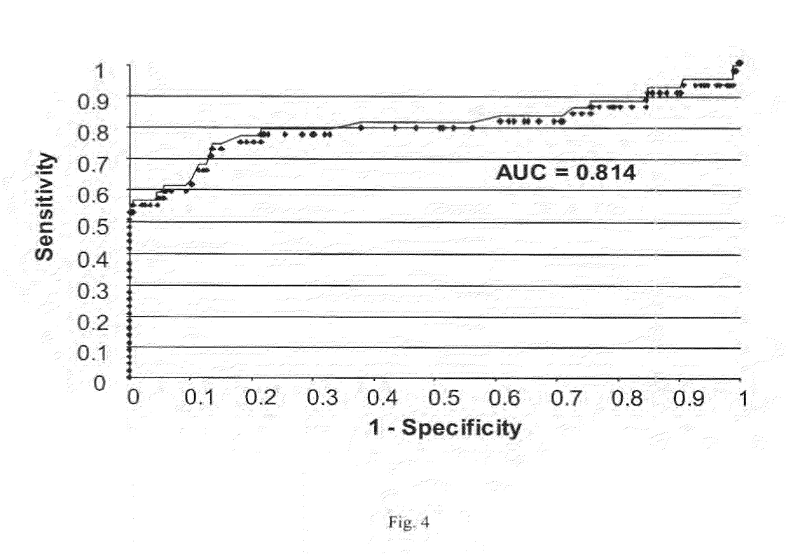

The third patient was an 84-year-old man, non-smoker who had suffered ischemic stroke on May 28-29, 2002 after angioplasty. He had receiving coumadin, cardizem, atenolol, hytrin, lasix, and guaifinex when he entered the study. NR2 antibodies measured at base line showed high antibody values above the cut off of 2 ng/ml (FIG. 3). Repeated blood tests revealed a trend of increasing NR2 antibodies that indicated a high risk of repetitive stroke. Oral Trental (200 mg×3 times a day) was initiated for 60 days and reduced the level of NR2 antibodies to the cut off. Antibody levels remained below the cut off level up to 1 year after the therapy.

### Example 3

### NMDA Receptor Peptide and Antibodies for Assessing Pulmonary System in Neonates with Congenial Heart Diseases Requiring Cardiac Surgery

Thirteen infants undergoing cardiac surgery (study group) were prospectively studied. NR2/NR3 peptide and antibodies were detected in all patients at 2 time points; (1) baseline before surgery; (2) 24-hours postop. Data obtained were compared to NR2/NR3 peptide/antibodies in normal infants (n=4, control group). The normal infants were having elective surgery for hernia or congenital cataracts. The average age was 1.6±2.3 months in the study group compared to 2.8±1.7 months in the control group control group. Cardiac operations included: TOF repair (3); ASO with VSD repair (1); ASO (1); CAVC repair (1); TOF/PA complete repair (1); TAPVR repair (1); hypoplastic

TABLE 4
NR2/NR3 peptide/Ab levels at baseline and 24 hours after cardiac surgery

| | | | | |
|---|---|---|---|---|
| Operation | Baseline | Baseline | 24 hr postop | 24 hr postop |
| | NR2/NR3 | NR2/NR3 Ab | NR2/NR3 | NR2/NR3 Ab |
| | (ng/ml) | (ng/ml) | (ng/ml) | (ng/ml) |

aortic arch repair (1): DCRV and VSD repair (1); coarctation repair and PAB (1): coarctation repair (1); Norwood (1). Baseline NR2/NR3 antibody levels were significantly higher in the study group compared to the control group (NR2/NR3 antibody p=0.002). NR2/NR3 peptide and antibodies levels at 24 hrs postop were statistically different compared to control group (NR2/NR3 peptide p=0.002; NR2/NR3 antibodies p=0.006). See Tables.

These data suggests that infants with congenital heart disease have elevated NR2/NR3 peptide and antibody at baseline compared to normal infants. This supports speculation that infants with congenital heart disease may have brain ischemia/injury prior to surgical intervention.

| | | | | |
|---|---|---|---|---|
| CAVC repair | 0.6 | 1.54 | 0.2 | 1.22 |
| PA/VSD repair | 0.2 | 1.8 | 0.4 | 1.8 |
| TAPVR repair | 0.2 | 1.11 | 1.1 | 1.22 |
| TOF remir | 1.2 | 1.66 | 0.9 | 1.57 |
| ASO/VSD repair | 1.3 | 1.66 | 1.2 | 1.62 |
| ASO | 8.9 | 1.5 | 2.2 | 1.53 |
| TOF repair | 0.2 | 1.46 | 0.6 | 1.52 |
| Aortic arch repair | 0.4 | 1.55 | 1.6 | 1.6 |
| DCRV/VSD repair | 1 | 1.2 | 0.8 | 0.8 |
| Coarctation | 0.4 | 0.6 | 0.2 | 0.5 |
| repair/PAB | | | | |
| Coarctation repair | 2.3 | 1 | 1.9 | 0.6 |
| Norwood | 0.3 | 0.7 | 1.3 | 1.9 |
| TOF repair | 0.26 | 0.48 | 0.1 | 0.30 |

Fallot; ASO = arterial switch operation;
DCRV = double chamber right ventricle;
PAB = pulmonary artery band
CAVC = complete atrioventricular canal;
PA = pulmonary atresia;
VSD = ventricular septal defect;
TAPVR = total anomalous pulmonary venous return;
TOF = tetralogy of

TABLE 5
NR2/NR3 peptide/Ab at baseline and 24 hours following cardiac surgery

| | NR2/NR3 peptide | NR2/NR3 antibody |
|---|---|---|
| | ng/ml | ng/ml |
| Control Group, N = 4 | 0.23 ± 0.13 | 0.73 ± 0.15 |
| Study Group | 1.33 ± 0.36 | 1.25 ± 0.44* |
| Baseline, N = 13 | | |
| Study Group | 0.96 ± 0.67^{#} | 1.24 ± 0.53^{#} |
| 24 hrs Postop, N = 13 | | |

*p < 0.05: control group vs baseline congenital heart disease
^{#}p < 0.05: control group vs 24 hour postop 2-tailed T-test

### Example 4

### Performance Characteristics of C1S-LA Antibody Test

Adult patients scheduled tor CPB (cardio-pulmonary bypass) surgery were evaluated for recombinant NR2 antibody levels in serum before, and subsequently evaluated for neurological complications within 48 hours after the surgery. Table 6 presents the pre-op NR2 Ab at different cut offs depending on post-operative adverse event. The neurological adverse events group included patients with confusion, TIA and stroke (N1HSS of >9 scores). Patients who had no neurological event were assigned to the group of entitled “No Neuro Event”.

TABLE 6
Neurological Complications (TIA/Stroke) Pre-Op or within
48 Hours vs. Pre-op NR2 Ab

| | | Neuro Event | No Neuro Event |
|---|---|---|---|
| Pre-Op NR2 Ab | | n/N (%) | n/N (%) |
| <1.5 | ng/mL | 7/213 (3.3%) | 206/213 (96.7%) |
| 1.5 to <2.0 | ng/mL | 12/159 (7.6%) | 147/159 (92.5%) |
| ≧2.0 | ng/mL | 25/26 (96.2%) | 1/26 (3.9%) |

Table 7 shows detailed analyses of six different cut offs for NR2 Ab concentrations from 1.5 to 2.0 ng/ml. Although the event rate increases in the cut offs from 1.5 to 2.0 for both groups, it increases faster in the “Neuro Event” group. Therefore, the risk ratio increases significantly over the analyzed range with the best risk ratio corresponding to 2.0 ng/mL (ClinChem, 2003). 96.0% (24/25) of patients with NR2 Ab concentrations ≧2.0 ng/ml preoperatively had neurological complications within 48 hours post-CPB. vs. only 5.4 % of patients with NR2 Ab concentrations <2.0 ng/ml. resulting in a 17.9-fold increase (95% C1, 11.6-27.6) in the predictive ability of a postoperative neurological adverse events.

TABLE 7
NR2 Ab results subdivided by different cut-offs

| | | | | Risk Ratio¹ |
|---|---|---|---|---|
| | | Neuro Event | No Neuro Event | (Lower 95% |
| Pre-Op NR2 Ab | | n/N (%) | n/N (%) | Bound)² |
| <1.5 | ng/mL | 8/214 (3.7%) | 206/214 (96.3%) | 5.2 |
| ≧1.5 | ng/mL | 36/184 (19.6%) | 148/184 (80.0%) | (2.8) |
| | | | | re 3 -- |
| <1.6 | ng/mL | 10/288 (3.5%) | 278/288 (96.5%) | 8.9 |
| ≧1.6 | ng/mL | 34/110 (30.9%) | 76/110 (69.1%) | (5.1) |
| <1.7 | ng/mL | 12/319 (3.8%) | 307/319 (96.2%) | 10.8 |
| ≧1.7 | ng/mL | 32/79 (40.5%) | 47/79 (59.5%) | (6.4) |
| <1.8 | ng/mL | 17/351 (4.S%) | 334/351 (95.2%) | 11.9 |
| ≧1.8 | ng/mL | 27/47 (57.5%) | 20/47 (42.6%) | (7.6) |
| <1.9 | ng/mL | 19/364 (5.2%) | 345/364 (94.8%) | 14.1 |
| ≧1.9 | ng/mL | 25/34 (73.5%) | 9/34 (26.5%) | (9.4) |
| <2.0 | ng/mL | 20/373 (5.4%) | 353/373 (94.6%) | 17.9 |
| ≧2.0 | ng/mL | 24/25 (96.0%) | 1/25 (4.0%) | (12.4) |

¹Ratio of Event rate among patients with a positive pre-op NR2 Ab divided by event rate among patients with a negative pre-op NR2 Ab;
²Lower one-sided 95% confidence bound on the risk ratio.

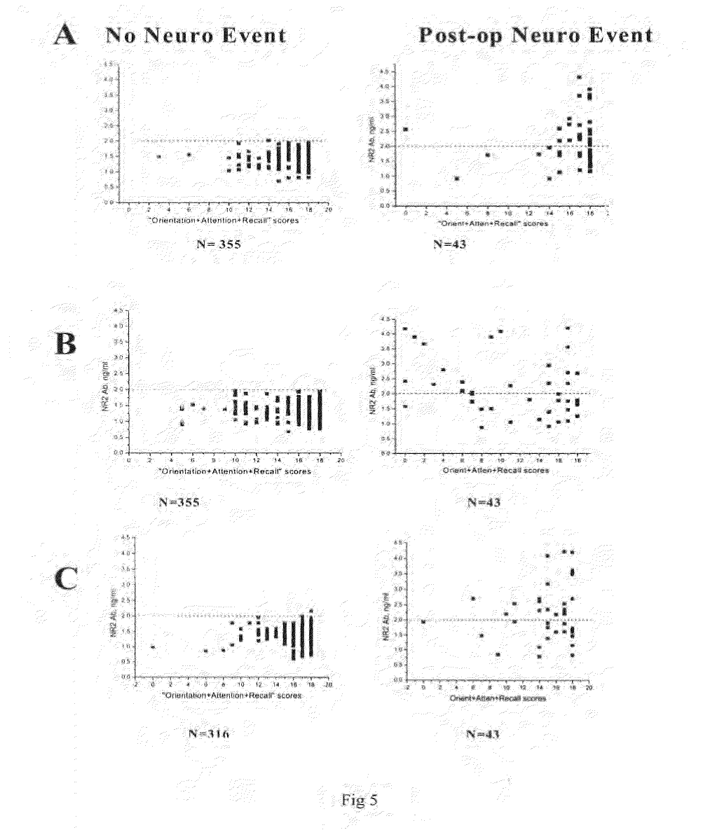

Based on the obtained likelihood ratio of a neuro event of 17.9, a NR2 antibody concentration exceeding 2.0 ng/ml detected pre-operatively can predict neurological complications in 89% of patients after surgery. FIG. 4 presents three ROC curves, treating the pre-op tests as diagnostics to create the ROC curve. The area under the curve for pre-op N2 Ab indicates high predictive ability (AUC=0.814) for neurological adverse events before surgery. Preoperative NR2 Ab had high predictive value for TIA/stroke before CPB. The concentrations of NR2 Ab in serum samples from patients with no adverse events (neurocode 0) maintained under the cut off of 2.0 ng/ml at all time points of the study. In contrast, most patients with neurological adverse events (N1HSS scores >9) had increased NR2 Ab values above the cut off of 2.0 ng/ml pre-op, 24 hours and 48 hours after the procedure.

Throughout this application, various publications are referenced. The disclosures of these publications in their entireties are hereby incorporated by reference into this application in order to more fully describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-42. (canceled)

43) An isolated peptide comprising SEQ ID NO:1, SEQ ID NO:2, SEQ ID NO:3 or SEQ ID NO:4, or an analog thereof.

44) The peptide of claim 43 linked to a diagnostic substrate or indicator reagent.

45) The peptide of claim 43 comprising SEQ ID NO:1.

46) The peptide of claim 43 comprising SEQ ID NO:2.

47) The peptide of claim 43 comprising SEQ ID NO:3.

48) The peptide of claim 43 comprising SEQ ID NO:4.

49) An isolated nucleic acid that encodes for a peptide comprising SEQ ID NO:1, SEQ ID NO:2, SEQ ID NO:3 or SEQ ID NO:4, or an analog thereof.
